(19) United States
(12) Patent Application Publication
(10) Pub. No.: US 2026/0069869 A1
(43) Pub. Date: Mar. 12, 2026

(54) PROGRAMMER AND ANTENNA FOR MEDICAL DEVICE

(71) Applicant: CVRx, Inc., Minneapolis, MN (US)

(72) Inventors: Bart Carey, Maplewood, MN (US); Clifford Rockwell, Maple Grove, MN (US); Morgan Beeson, Plano, TX (US); Chris Story, Fort Worth, TX (US)

(21) Appl. No.: 19/110,528

(22) PCT Filed: Sep. 12, 2023
(86) PCT No.: PCT/US2023/073995

§ 371 Date: Mar. 11, 2025

Related U.S. Application Data
(60) Provisional application No. 63/405,648, filed on Sep. 12, 2022.

(51) Int. Cl. A61N 1/372

(52) U.S. Cl. CPC A61N 1/37247

(57) ABSTRACT

Methods and associated devices for programming a therapy for an implantable baroreflex activation system are described, including for programming the implantable baroreflex stimulation system with operating parameters.

### RELATED APPLICATIONS

This application claims the benefit of U.S. Provisional Application No. 63/405,648, filed Sep. 12, 2022, the disclosure of which is incorporated by reference herein.

### TECHNICAL FIELD

Embodiments of the present disclosure relate generally to implantable baroreflex activation therapy devices, and more particularly to devices and methods for programming or adjusting operating parameters of such therapy devices.

### BACKGROUND

Cardiovascular disease is a major contributor to patient illness and mortality, and is also a primary driver of health care expenditure. Heart failure is the final common expression of a variety of cardiovascular disorders, characterized by an inability of the heart to pump enough blood to meet a patient's needs. Symptoms of heart failure include fatigue, reduced exercise capacity and poor survival.

Hypertension, or high blood pressure, is a major cardiovascular disorder that is estimated to affect tens of millions of people in the United States alone. Hypertension is a leading cause of heart failure and stroke, is the primary cause of death for tens of thousands of patients per year, and is listed as a primary or contributing cause of death for hundreds of thousands of patients per year in the United States alone.

Accordingly, heart failure and hypertension are serious health problems demanding significant research and development for the treatment thereof.

One method of treating hypertension and/or heart failure is baroreflex activation therapy (or “BAT”), which comprises stimulation of baroreceptors and/or associated nerves or nerve structures. Baroreceptors are sensory nerve ends that are profusely distributed within the walls of the major arteries, as well in the heart, aortic arch, carotid sinus or arteries, and in the low-pressure side of the vasculature such as the pulmonary artery and vena cava. Baroreceptor signals are used to activate a number of body systems which collectively may be referred to as the baroreflex system. Baroreceptors are connected to the brain via the nervous system. Thus, the brain is able to detect changes in blood pressure, which can be related to, or indicative of, cardiac output.

Activating the baroreflex (by, for example, stimulation of baroreceptors) increases afferent electrical signals from the source (baroreceptor) through the carotid sinus nerve (Hering's nerve, a branch of the glossopharyngeal nerve, cranial nerve IX) to the medullary brain centers that regulate autonomic tone. Increased afferent signals to these medullary centers cause a reduction in sympathetic tone and an increase in parasympathetic tone. This results in lower heart rate, reduced sodium and water reabsorption by the kidney resulting in a diuresis, relaxation of the smooth muscle in the blood vessels which results in vasodilatation and a reduction in blood pressure. Thus, peripheral activation of the baroreflex results in a physiologic response whereby blood pressure is controlled by mechanisms determined by the integrative action of the central nervous system action on all peripheral organs and blood vessels.

Baroreflex activation therapy works by artificially activating the carotid sinus baroreflex. U.S. Pat. No. 6,522,926 to Kieval, et al. discloses a baroreflex activation system and method for activating baroreceptors to regulate blood pressure for the treatment of hypertension and/or heart failure (to counteract the above-described pressor response). Generally speaking, the baroreceptor activation device may be activated, deactivated or otherwise modulated to activate one or more baroreceptors and induce a baroreceptor signal or a change in the baroreceptor signal to thereby affect a change in the baroreflex system. The baroreceptor activation device may be activated, deactivated, or otherwise modulated continuously, periodically, or episodically. The baroreceptor activation device may utilize electrical, mechanical, thermal, chemical, or biological means, or a combination thereof to activate the baroreceptor. The baroreceptor may be activated directly, or activated indirectly via the adjacent vascular tissue.

Current generation implantable baroreflex therapy systems feature implantable pulse generators and therapy electrodes with reduced form factors as compared to early systems. One such system, described in U.S. Pat. No. 8,437,867 to Murney et al., includes an implantable pulse generator and associated circuitry contained within a hermetically sealed housing, an elongate flexible electrical lead connectable to the housing, and a monopolar electrode structure coupled with the electrical lead. Current generation BAT systems offer improvements to the implant procedure, and can be implanted via a minimally invasive approach.

After the device has been successfully implanted, a number of operating parameters may be adjusted and/or tested in order to tailor operation of the device to the particular patient. Ideally, the operating parameters of the device are adjusted to provide a desired therapeutic effect, while minimizing any side effects attributable to operation of the device. Further, because the device includes a dedicated power supply having a finite energy amount, it is desirable to adjust the operating parameters to prolong the service life of the device to avoid the cost and trauma associated with replacing a battery of a device (or entire device) due to a depleted power supply. Programming the device, therefore, involves a tradeoff between obtaining a desired response, minimizing or preventing side effects, and prolonging the service life of the power supply of the device.

An opportunity exists to improve upon current programming devices and techniques to provide simpler, faster, safer and/or more effective methods of programming an implanted baroreflex activation device for delivery of chronic therapy.

### SUMMARY

The techniques of this disclosure generally relate to devices and methods for programming a therapy having at least two programmable operating parameters for an implanted baroreflex stimulation system that is configured to deliver the therapy to a patient.

In one aspect, a programming system for programming the therapy includes a computing device configured to present a user interface for adjusting the at least two programmable operating parameters, a programing device communicatively coupled to the computing device and the implanted baroreflex stimulation system, and a case configured to receive the computing device. The programming device has a body pivotally coupled to a base including a protrusion, and a button configured to, when pressed, apply force adjacent to and towards the protrusion. The case has one or more receiving components configured to removably receive the protrusion, such that the computing device and the programming device are coupled until the button is pressed.

In embodiments, the programming device further includes an aperture configured to allow the programming device to be hung, such as from an IV pole or similar apparatus.

In a second aspect, a method of programming a therapy having at least two programmable operating parameters for an implanted baroreflex stimulation system, the implanted baroreflex stimulation system configured to deliver the therapy to a patient, includes presenting a user interface via a computing device. The user interface includes a main page displaying patient information, therapy status, battery status, lead impedance, radio frequency (RF) signal strength, a graph of lead impedance trend of the implanted baroreflex stimulation system, and the at least two programmable operating parameters of the therapy. The method further includes displaying a second page including an option to adjust each of the at least two programmable operating parameters, receiving user input to adjust one or more of the at least two programmable operating parameters, and determining compliance of the implanted baroreflex stimulation system operating with the adjusted one or more programmable operating parameters. In response to a determination of compliance, the method includes programming the implanted baroreflex stimulation system to use the adjusted one or more programmable operating parameters.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is a perspective view of a system for programming a baroreflex activation device according to an embodiment.

FIG. 2A is a perspective view of the system of FIG. 1 in a disconnected, standing arrangement according to an embodiment.

FIGS. 2B, 2C, and 2D are perspective views of the system of FIG. 2A in a disconnected, hanging arrangement.

FIG. 2E is a perspective view of the system of FIG. 2A in a disconnected, handheld arrangement.

FIG. 3A is a perspective view of a programmer for programming a baroreflex activation device according to an embodiment.

FIG. 3B is a perspective, cross-sectional view of the programmer of FIG. 3A.

FIG. 4A is a rear view of a programmer for programming a baroreflex activation device according to an embodiment.

FIG. 4B is a rear view of the programmer of FIG. 4A.

FIG. 5A is a perspective view of a computing device for programming a baroreflex activation device according to an embodiment.

FIG. 5B is a close-up, partial view of the computing device of FIG. 5A.

FIG. 5C is a top-down view of the computing device of FIG. 5A.

FIG. 5D is a side view of the computing device of FIG. 5A.

FIG. 6 is a flowchart of a method for programming a baroreflex activation device according to an embodiment.

FIG. 7A is a main operational page of a user interface for a programmer according to an embodiment.

FIG. 7B is a main operational page of a user interface for a programmer according to an embodiment.

FIG. 7C is a parameter adjustment page of a user interface for a programmer according to an embodiment.

FIG. 7D is a parameter adjustment page with an amplitude adjustment slider of a user interface for a programmer according to an embodiment.

FIG. 7E is a lead impedance trend page of a user interface for a programmer according to an embodiment.

FIG. 7F is a patient selection page of a user interface for a programmer according to an embodiment.

FIG. 8 is a flowchart of a method of programming a baroreflex activation device according to an embodiment.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

The following detailed description should be read with reference to the drawings in which similar elements in different drawings are numbered the same. The drawings, which are not necessarily to scale, depict illustrative embodiments and are not intended to limit the scope of the invention.

An implanted baroreflex system can be activated to improve cardiac function and reduce excessive blood pressure, autonomic nervous system activity and neurohormonal activation to address the problems of heart failure, hypertension, other cardiovascular disorders and renal disorders. In particular, baroreceptors may be activated, thereby indicating an increase in blood pressure and signaling the brain to reduce the body's blood pressure and level of sympathetic nervous system and neurohormonal activation, and increase parasympathetic nervous system activation, thus having a beneficial effect on the cardiovascular system and other body systems.

For general information pertaining to the cardiovascular, circulatory, and nervous systems, as well as baroreceptor and baroreflex therapy systems that may be used in whole or in part with embodiments of the present invention, reference is made to the following commonly assigned patent applications and patents: US Published Patent Application Nos. 2006/0004417 to Rossing et al., 2006/0074453 to Kieval et al., 2014/0277279 to Cates et al., and 2021/0316135 to Pignato et al., and U.S. Pat. Nos. 6,522,926, 6,850,801, 6,985,774, 7,480,532, 7,499,747, 8,249,705, and 8,620,422 all to Kieval et al., U.S. Pat. No. 7,499,742 to Bolea et al., U.S. Pat. No. 7,835,797 to Rossing et al., U.S. Pat. No. 7,848,812 to Crowley et al., U.S. Pat. No. 8,086,314 to Kieval, and 8,150,521 to Crowley et al., U.S. Pat. No. 8,437,867 to Murney et al., U.S. Pat. No. 9,345,877 to Pignato et al., the disclosures of which are hereby incorporated by reference in their entireties except for the claims and any express definitions.

After a baroreflex activation system has been successfully implanted, a physician or other medical professional may utilize a programming device to enable operation of the system and to interrogate, adjust, or monitor the therapies being delivered by the implanted baroreflex activation system. Further, the programming device may be used to monitor status information of the implanted baroreflex activation system including battery voltage or end of service indicators. In an embodiment, the programming device may be utilized to adjust one or more operating parameters of the baroreflex activation system to suit the particular patient. Operating parameters may also be referred to as regimen parameters, therapy parameters, therapy characteristics, signal characteristics, pulse characteristics or other similar terms as will be apparent to one skilled in the art for describing parameters or characteristics which impact or effect therapy efficacy and/or device longevity. By focusing on adjusting one or more of pulse amplitude, pulse width and pulse frequency, a suitable stimulus regimen can be created for the particular patient and saved within the implanted control system.

For a given baroreflex activation system, the pulse amplitude at a given pulse width is constrained by impedances of the electrode, the lead, internal circuitry of the implantable pulse generator, or other factors, thereby creating an upper limit to suitable pulse amplitude values. Similarly, the anatomy of each individual patient and the placement of the implanted electrode will limit the available combinations of pulse amplitude and pulse width which can be used. Exceeding these limits can create extraneous stimulation (e.g., stimulation of other nerves, nerve fibers, or other excitable tissues) which not only reduces efficacy of the baroreflex therapy but typically creates pain or discomfort to the patient. Finally, because some implanted baroreflex activation systems are powered by a dedicated battery having a finite energy amount, it may be desirable to prolong the service life of the system as much as practical, to avoid or delay the cost and trauma associated with replacing a system having a depleted power supply. While a stimulus regimen programmed with relatively large pulse amplitudes generally results in a greater therapeutic effect (i.e., efficacy), such large pulse amplitude signals generally result in decreased battery life.

Therefore, a suitable stimulus regimen is one which is within the compliance limits of the characteristics of the baroreflex activation system, which avoids patient discomfort resulting from extraneous tissue stimulation, and which maintains acceptable battery life.

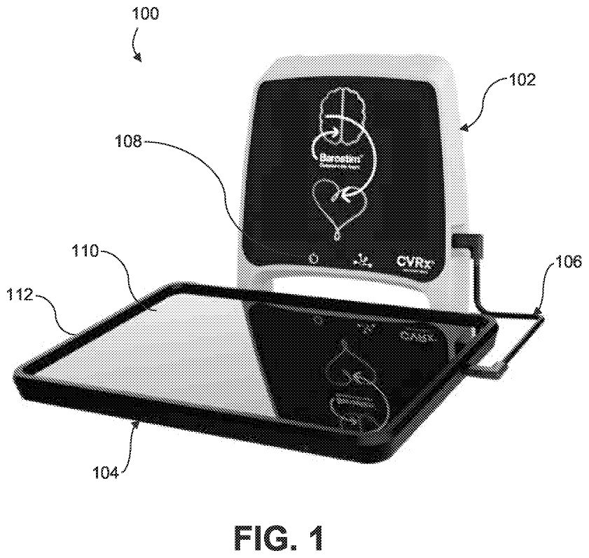

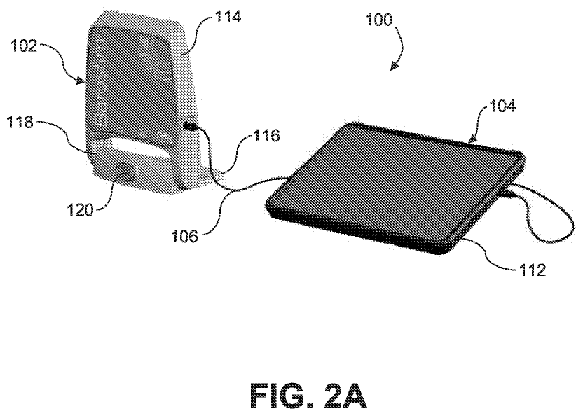

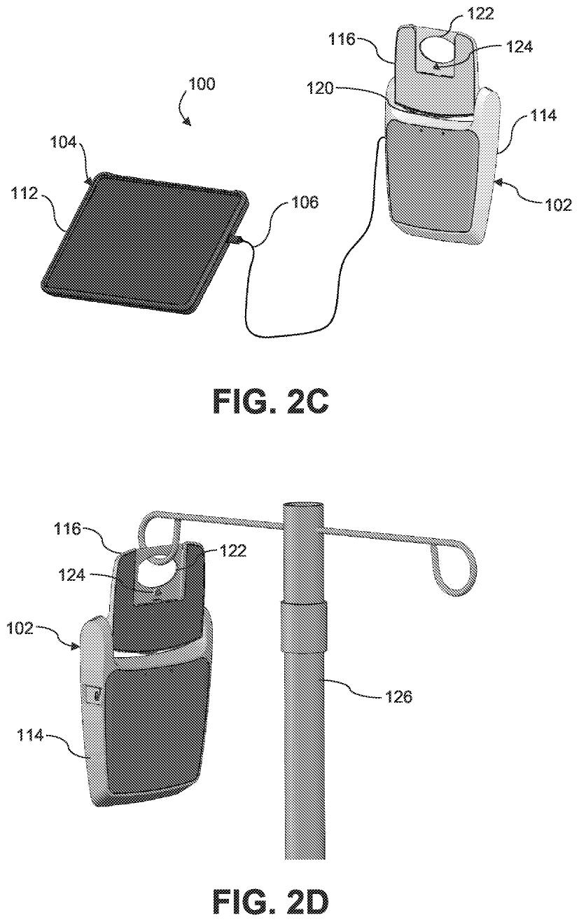

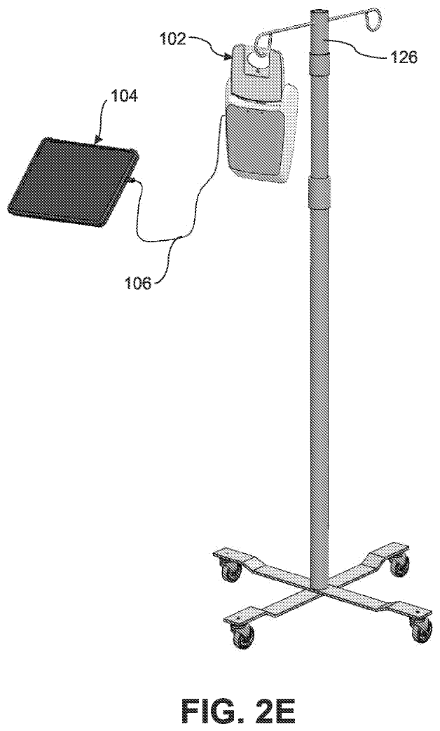

Referring now to FIGS. 1-2E, a system 100 for programming a baroreflex activation device is depicted according to embodiments. System 100 generally comprises programmer 102 and computing device 104. Programmer 102 and computing device 104 are communicatively coupled via cable 106 such that computing device 104 can provide input and display capability to programmer 102. In embodiments, programmer 102 and computing device 104 can communicate wirelessly, such as via Bluetooth, or with multiple cables in place of, or including, cable 106.

Programmer 102 is an external programming device, and may also be referred to as a programmer interface. In embodiments, programmer 102 can be configured to receive data (e.g., device operational data, feedback from sensors, diagnostics data, therapeutic delivery data, efficacy data, etc.). In an embodiment, programmer 102 may include one or more telemetry links for communicating with a baroreflex activation device or computing device 104. Programmer 102 includes an antenna configured for communication.

Computing device 104 may be a mobile cellular device, desktop computer, laptop, tablet, smart watch or other wearable, dedicated programming device, or combinations thereof. Computing device 104 includes a user interface 110 such as touchscreen or keypad. In embodiments, computing device 104 is stored within case 112 that is configured to removable couple to programmer 102 for ease of use as will be described later in greater detail. As depicted in the Figures, computing device 104 comprises a tablet. In embodiments, computing device 104 provides power to programmer 102 via cable 106.

In embodiments, programmer 102 can be battery powered and operated via a Bluetooth connection to computing device 104. In such arrangements, other connection means such as Wi-Fi are also contemplated. By reducing, or removing, the need for cable 106, computing device 104 can have improved flexibility of use.

Programmer 102 and computing device 104 may be configured to wirelessly communicate with one or more of a baroreflex activation device, a related accessory, or each other. In embodiments, programmer 102 and computing device 104 may be physically connected to a baroreflex activation device via one or more cables. In embodiments, system 100 can be configured to transmit programming data or instructions.

In embodiments, a carrier (e.g., a bag, backpack, tote or the like) may optionally be included as part of system 100. The carrier may be configured to receive and hold programmer 102 and computing device 104 in a convenient, consolidated manner, and offer portability of system 100. In embodiments, the carrier may include a control or operation interface, in place of or in addition to, computing device 104.

In some embodiments, data communicated between programmer 102 and a baroreflex activation device can be transmitted to an optional external server for analysis, storage, wider dissemination, or similar uses. In some embodiments, the external server can be configured as a network of servers and/or a computing cloud. For example, in some embodiments, the external server can include one or more complex algorithms representing machine learning and/or a neural network configured to process and analyze data to further improve patient outcomes. In embodiments, data from one or more of programmer 102 and the external server may be communicable with, or accessible by, a clinician.

Data from one or more sensors, or auxiliary sensors, associated with a baroreflex activation device may be communicated with programmer 102. In embodiments, operation of system 100 may be modified based on data from one or more sensors or auxiliary sensors.

Closed loop operation of system 100 may include a manual override. For example, cessation of closed loop operation may be important in the event that a physiologic sensor is compromised or if conditions arise when manual coordination of therapy becomes important (e.g., during specific events such as percutaneous coronary intervention for example). In an embodiment, programmer 102 may be configured to override closed loop operation.

In an embodiment of open loop operation, programmer 102 may display one or more operating parameters of system 100, allowing a user or clinician to modify one or more parameters. Examples of parameters adjustable using programmer 102 may include output amplitude, duration, frequency, or polarity.

Programmer 102 generally comprises body portion 114 and base portion 116 that are pivotally coupled via hinges 118. Base portion 116 is configured to removably couple case 112 of computing device 104 in different arrangements. FIG. 2A depicts a disconnected (e.g., physically uncoupled), standing arrangement where computing device 104 can move independently from programmer 102. FIG. 1 depicts a connected (e.g., physically coupled), standing arrangement where case 112 of computing device 104 is coupled to base portion 116 as will be described later. Case 112 can be disconnected from base portion 116 by pressing button 120 on base portion 116. FIG. 2B depicts a disconnected, hanging arrangement whereby programmer 102 can be hung using aperture 122 in base portion 116. In operation, aperture 122 can be used to hang programmer 102 from IV pole 126 as depicted in FIGS. 2C-2D.

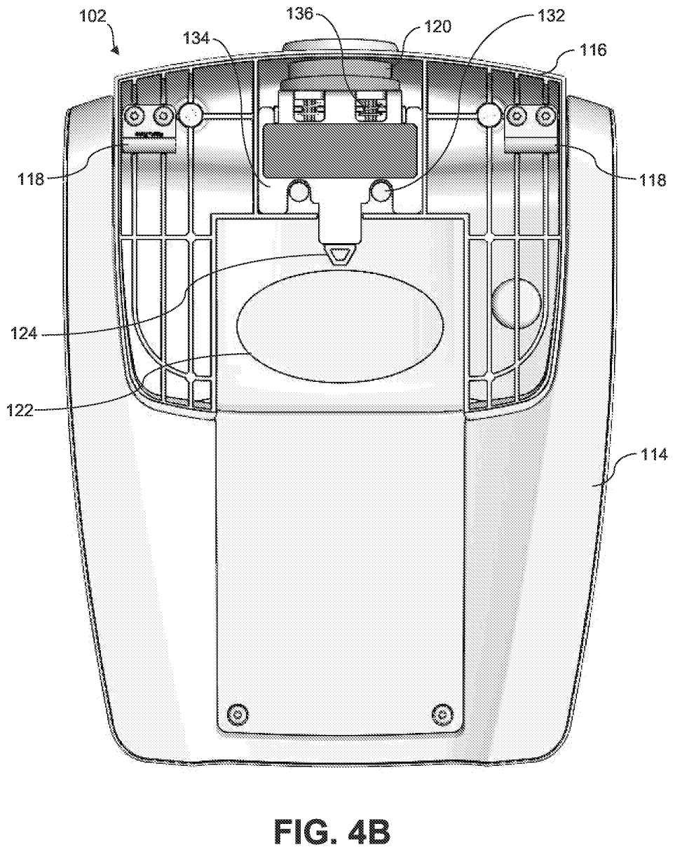

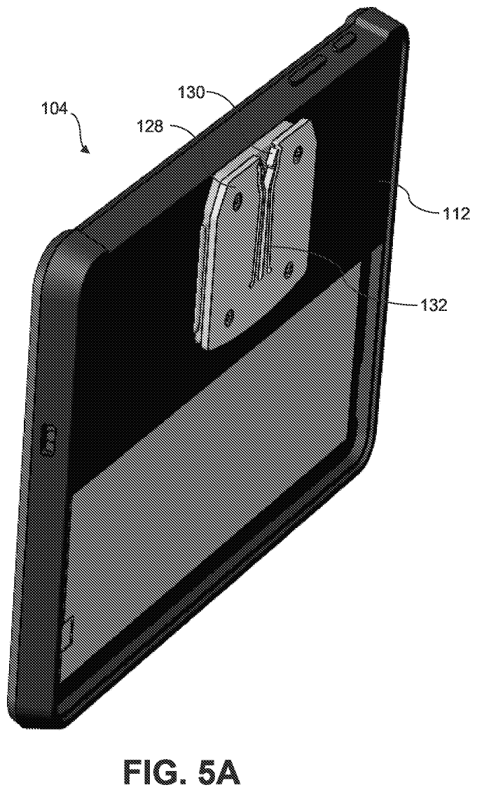

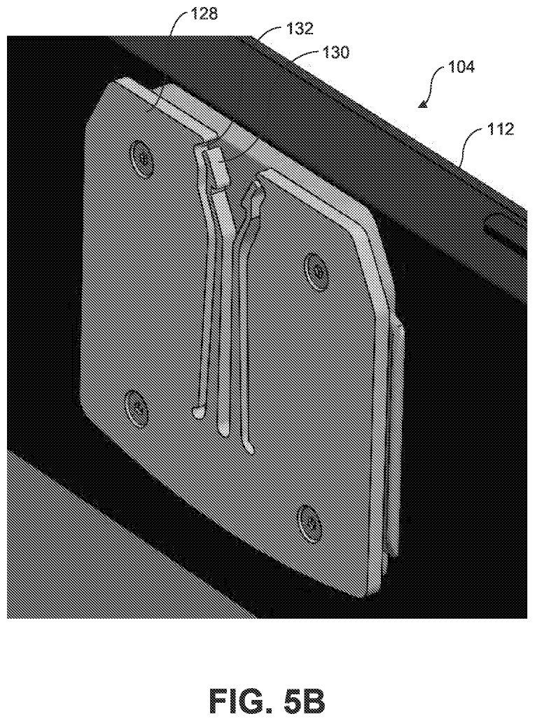

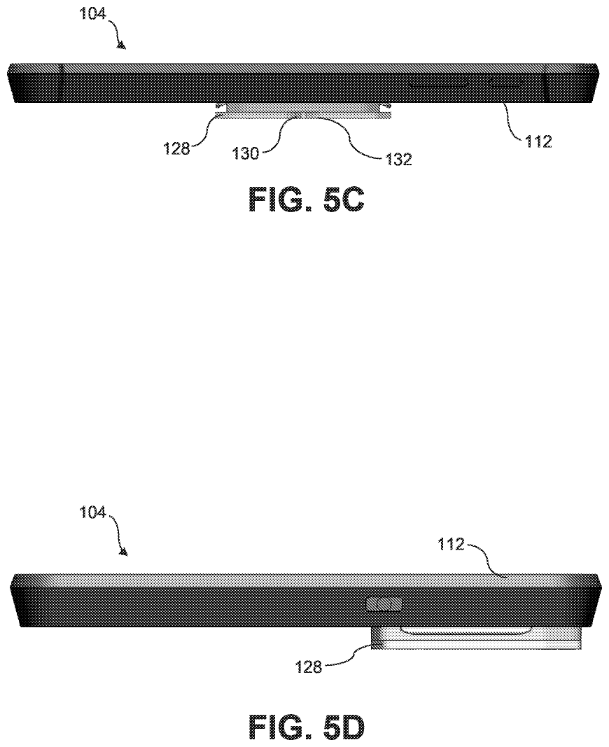

In embodiments, programmer 102 can be operably coupled to computing device 104 via one or more interlocking components 124 that can be selectively released by pressing button 120. In embodiments, one or more interlocking components 124 can be slid into or otherwise merged with one or more receiving components 128 of case 112, depicted in greater detail in FIG. 5B. In other embodiments, the case of computing device 104 can comprise interlocking components and the base portion 116 of programmer 102 can comprise one or more receiving components. The alignment of such a coupling is depicted in FIG. 2E, where system 100 is depicted in a disconnected, handheld arrangement. In the depicted embodiment, computing device 104 could be coupled to programmer 102 by inserting interlocking component 124 into receiving component 128. In embodiments, base 116 of programmer 102 can include one or more grips to prevent programmer 102 from sliding on a surface when in a standing arrangement or to reduce movement relative to computing device 104 when in a connected, handheld arrangement.

The versatile arrangements of programmer 102 and the reduced form factor resulting from the use of a mobile device, such as a tablet, as computing device 104, enables efficient storage of system 100 when not in use and adaptability to diverse clinical settings when in use. This form factor, particularly during storage or handheld use, is facilitated by the ability of programmer 102 and computing device 104 to rest flat against each other while coupled.

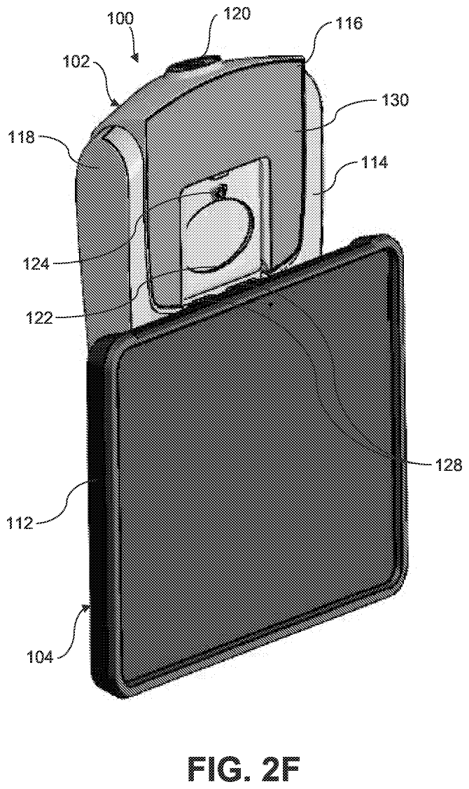

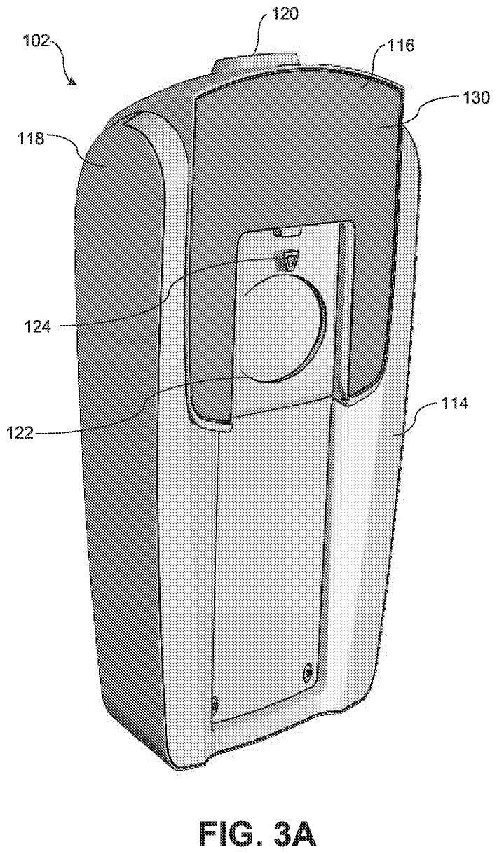

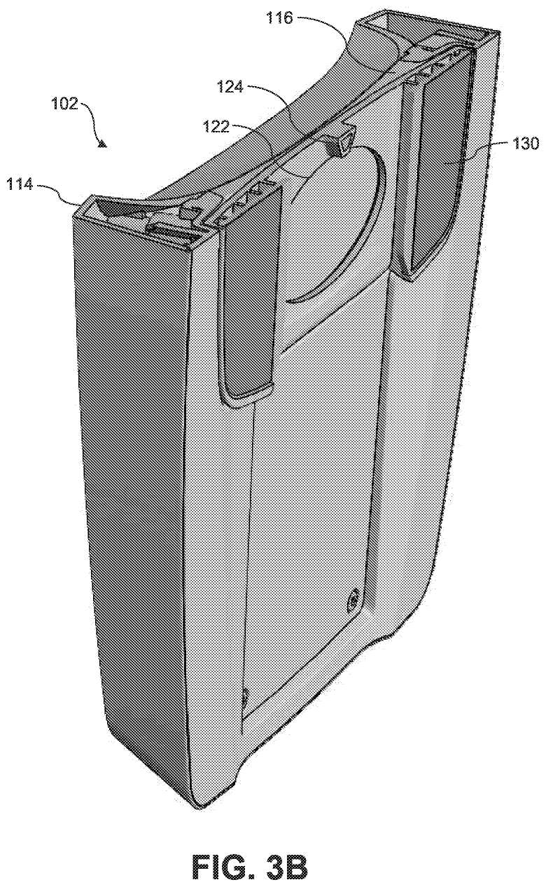

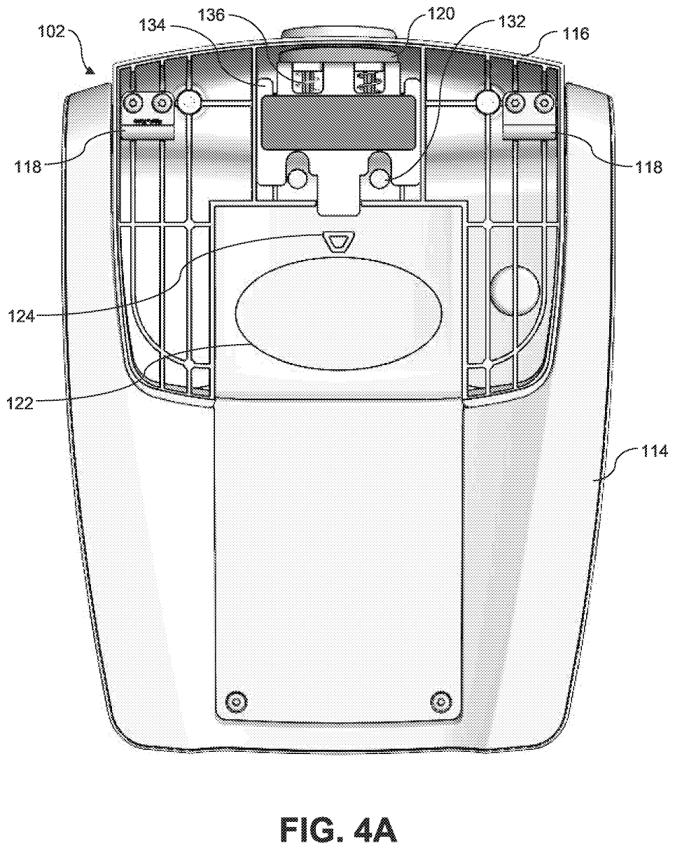

Referring now to FIGS. 3A-3B, programmer 102 is depicted in greater detail according to embodiments. FIG. 3B depicts a cross sectional view of programmer 102. A view of the interlocking mechanism of programmer 102 is shown in greater detail in FIGS. 4A-4B according to embodiments.

FIG. 4A depicts the interlocking mechanism of programmer 102 with button 120 not pressed. Button 120 is coupled to wedge 134 by springs 136 such that when button 120 is pressed, as shown in FIG. 4B, wedge 134 extends to interlocking component 124. If receiving component 128 of computing device 104 is coupled to interlocking component 124 of programmer 102 when button 120 is pressed, wedge 134 applies sufficient force to separate the components by pushing receiving component 128 away from interlocking component 124.

Referring now to FIGS. 5A-5D, computing device 104 is depicted within case 112 according to embodiments. Receiving component 128 comprises prongs 130 and channels 132. When connected to programmer 102, interlocking component 124 can be inserted into receiving component 128 such that prongs 130 are forced into channels 132 and thereafter biased against interlocking component 124. The connection can then be separated by pressing button 120 as described with respect to FIGS. 4A-4B.

The incorporation of receiving means 128 in case 112 enables the incorporation of existing tablets or other computing devices into system 100 without the need to produce a custom computing device. This flexibility allows different computing devices to be used by simply changing the size or layout of the case to match the computing device. In other embodiments, computing device 104 can comprise case 112 such that one or more elements of case 112, such as receiving component 128, are built into computing device 102.

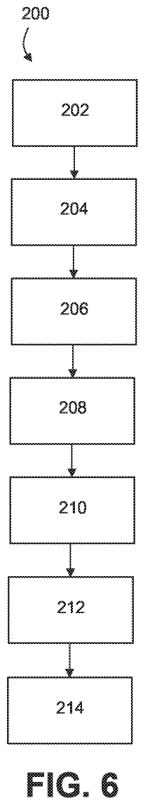

Referring now to FIG. 6, a method 200 of programming a baroreflex activation device is depicted which begins in optional step 202 by obtaining baseline patient hemodynamic measurements such as blood pressure (systolic, diastolic, peripheral or central), heart rate, respiration, posture, electrocardiogram, blood volumetric flow rate, blood flow velocity, blood pH, O₂ or CO₂ content, mixed venous oxygen saturation (SVO₂), vasoactivity, nerve activity, tissue activity, or other suitable parameters. Such baseline patient hemodynamic measurements may be obtained with programmer 102, or may be obtained by other means and the measurements manually entered into programmer 102 via computing device 104. In step 204, initial operating parameters are programmed into the baroreflex activation device. In step 206, the pulse amplitude is adjusted and set. In step 208, the pulse width is adjusted and set. In step 210, the pulse amplitude and pulse width settings are verified. In step 212, patient hemodynamic measurements are obtained. And in step 214, the pulse frequency is adjusted and set as needed.

The programmer system is configured to adjust any of the operating parameters described herein (such as pulse width, pulse amplitude, pulse frequency, etc.) as well provide an indication of the magnitude of the operating parameters; track the history of the operating parameters during the programming process; provide an indication of a patient response to a delivered therapy, such as by way of indicating an actual value for a patient physiological parameter or simply indicating favorable or unfavorable response; and provide indications of predicted battery life based on selected operating parameters. The programmer may prompt the user with step-by-step instructions to perform the programming steps as described herein, or the programming device may offer a manual operation mode, allowing a user to tailor the programming process as desired such as skipping any or all steps.

| Parameter | Description | Range of Values |
|---|---|---|
| Pathway | Determines the position of applied pulses for the therapy. | Left, Right, Both |
| Pulse Width | Determines the width of the applied pulse. Can be configured individually for | 15 microseconds to 500 |
| | the left and right pathways. | microseconds |
| Amplitude | Determines the amplitude of the applied pulses. Can be configured | 1.0 milliamps to 20.0 |
| | individually for the left and right pathways. | milliamps |
| Therapy Frequency | Determines the frequency of applied pulses except during the Rest portion of | 10 to 100 pulses per |
| | the Burst Interval. | second |
| Burst Enable | Determines whether therapy pulses are applied throughout the burst cycle in | On, Off |
| | a continuous manner or if a cycle of active and rest periods is being used. | |
| Burst Duration | Determines the length of the active portion of the burst cycle during which | 50 milliseconds to 1950 |
| | the Therapy Frequency is delivered. | milliseconds |
| | NOTE: This parameter is not shown if Burst Enable is Off. | |
| Burst Interval | Determines the total length of the burst cycle including the active portion and | 100 milliseconds to 2000 |
| | the rest portion. | milliseconds |
| | NOTE: This parameter is not shown if Burst Enable is Off. | |

In one embodiment, tracking the history of the operating parameters during the programming process may comprise plotting the parameters as they are tested, with for example a first operating parameter along a first axis and a second operating parameter along a second axis. In another embodiment, the programming device may store values from past programming sessions.

Referring now to an alternate embodiment of programming method 200, the order of steps 206 and 208 are reversed such that step 208 is carried out prior to step 206. In such an embodiment, step 208 comprises setting a nominal value for pulse amplitude, such as 0.8 mA or 1.0 mA, and then adjusting the pulse width according to the procedures described above. Subsequently, in step 206 the pulse amplitude is adjusted according to the procedures described above.

In an alternate embodiment, steps 206 and 208 are carried out simultaneously, such that if the patient is not experiencing extraneous stimulation, both pulse amplitude and pulse width are increased at the same time before continuing to deliver therapy signals and check for patient response. In such an embodiment, the width and amplitude are increased from the initial value in a predetermined ratio until extraneous is encountered or compliance is exceeded. The amplitude and width are then simultaneously reduced in a predetermined ratio to eliminate extraneous, stay within compliance limits, and provide reasonable longevity as determined by the user. The ratio may be in the range of 0.1-1 mA for every 1-15 us. A preferred ratio includes increments of 0.4 mA for every 5 us. This approach has the advantage of determining programmed parameters with only a single control.

In an alternate embodiment, pulse width is set to an estimated value, the estimated value being greater than the initial value described above, and pulse amplitude is then increased in increments as described in conjunction with step 206, such that step 208 is effectively eliminated. Steps 210, 212 and 214 are then carried out as described above.

In an alternate embodiment, pulse amplitude is set to an estimated value, the estimated value being greater than initial value described above, and pulse width is then increased in increments as described in conjunction with step 208, such that step 206 is effectively eliminated. Steps 210, 212 and 214 are then carried out as described above.

In another embodiment, the programming method may take into account projected device longevity (e.g., useful service life of the battery of the implantable pulse generator) when setting the various operating parameters. For example, programmer 102 is configured to calculate the projected or predicted device longevity based on one or more parameters including but not limited to battery capacity, pulse amplitude, pulse width, and pulse frequency. In one embodiment, programmer 102 may provide an indication to a user of projected device longevity during the programming procedure, such that the user may choose whether or not to modify an operating parameter based on the indication of device longevity provided by programmer 102.

In another embodiment, programmer 102 may provide the option to select a mode of operation which can increase device longevity, such as selecting to deliver burst therapy or periodic therapy, wherein therapy is delivered for a given period of time and then not delivered for a period of time before being delivered again. Such an arrangement may maintain suitable therapeutic effect of the delivered therapy while extending device longevity.

In another embodiment, the user may input a desired device longevity programmer 102. For example, the user may input a desired device longevity at the beginning of programming method 200, and then proceed with method 200 as described above. In conjunction with one or more steps of programming method 200, an indication of device longevity is provided to the user and may thus influence the selected parameters. In another example, the user inputs a desired device longevity at the beginning of programming method 200 and programmer 102 provides suggested values for one or more device operating parameters such as pulse amplitude, pulse width, pulse frequency, or others. The programming method may then begin by testing the suggested values based on the desired device longevity and then adjusted as needed.

It may be advisable when setting any of the operating parameters described herein to create a safety margin (factor) or buffer against extraneous stimulation, as the level at which a patient experiences extraneous stimulation may vary according to activity, posture, stress, environmental factors, physiological fluctuations or other factors. In one embodiment, the safety margin or buffer may be expressed as a percentage, such that if a patient experiences extraneous stimulation at a programmed value of X, the safety margin is defined as 90% of X (thereby creating a buffer of 10%). Alternate magnitudes for the buffer may be in the range of 5% to 25%.

Alternately, the safety margin or buffer may be expressed as a numerical value, such that if a patient experiences extraneous stimulation at a programmed value of Y, the safety margin is defined as Y minus Z, wherein Z is the buffer. In one embodiment, the buffer may be the same as the increments by which the various operating parameters are adjusted during programming. In one embodiment, the buffer may be based on historical data obtained from prior programming procedures for the same patient and/or other patients, and such historical data may be relied upon to set or at least influence the buffer setting for the present patient. Optionally, the system may recommend parameter settings for pulse width and/or pulse amplitude using extraneous and/or historically-obtained buffer data which simultaneously maximize proximity to the buffer (and therefore maximize effectiveness of therapy) and maximize device longevity.

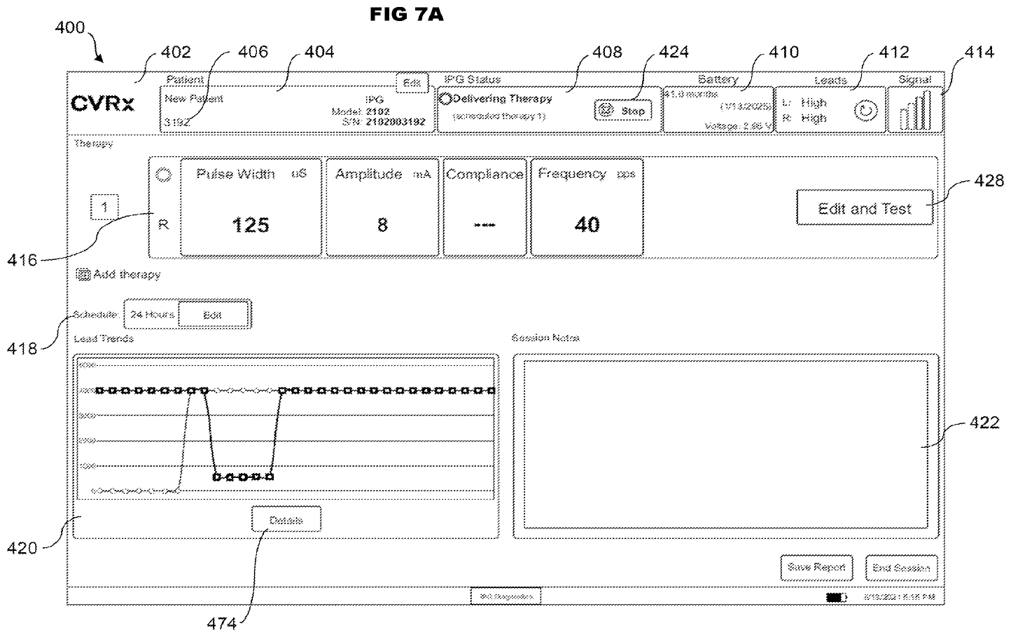

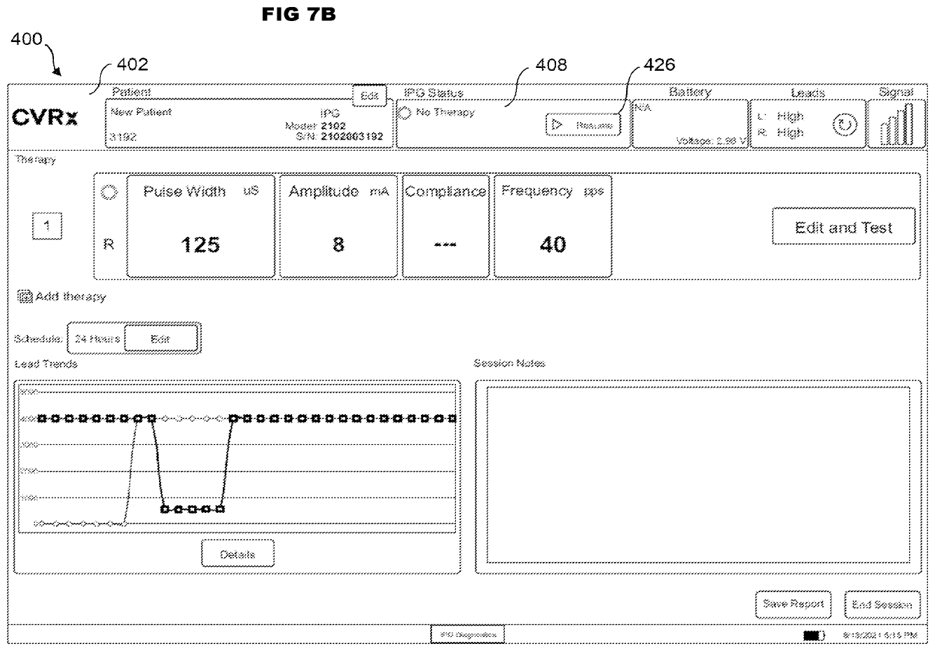

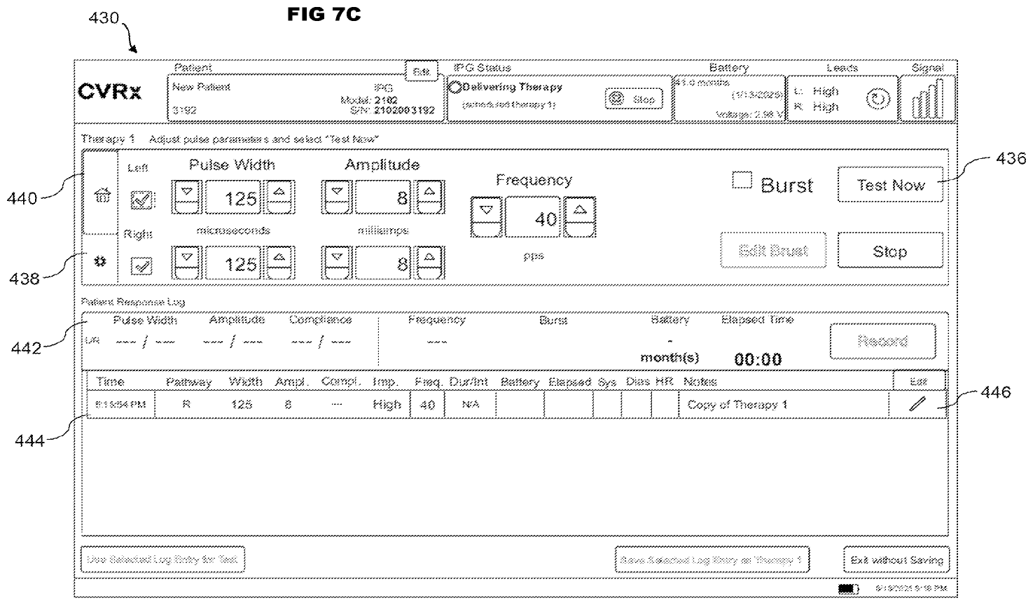

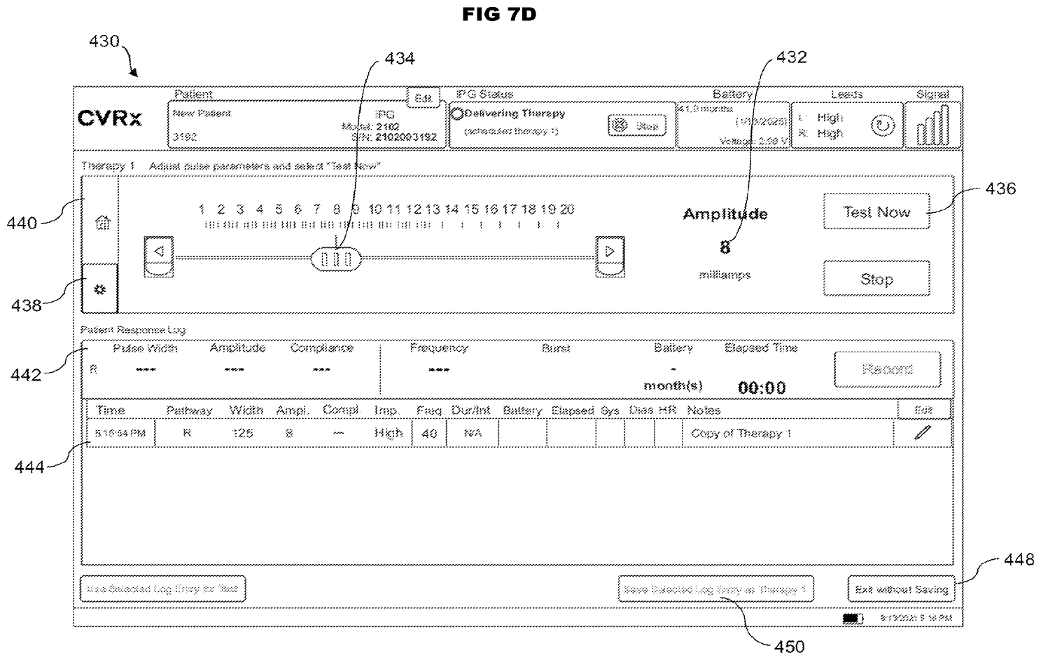

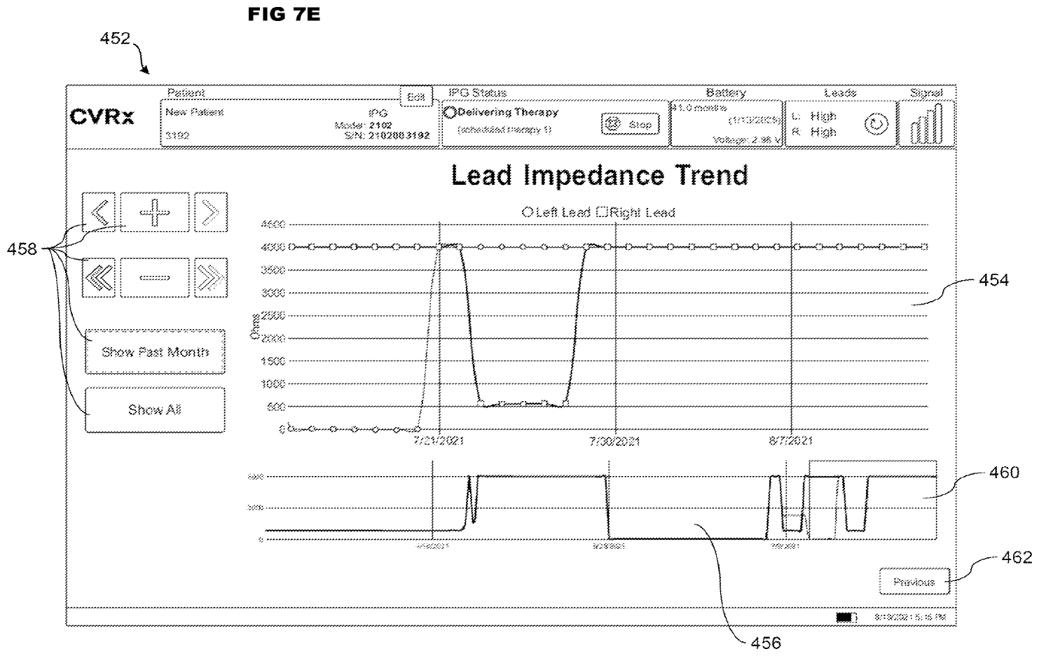

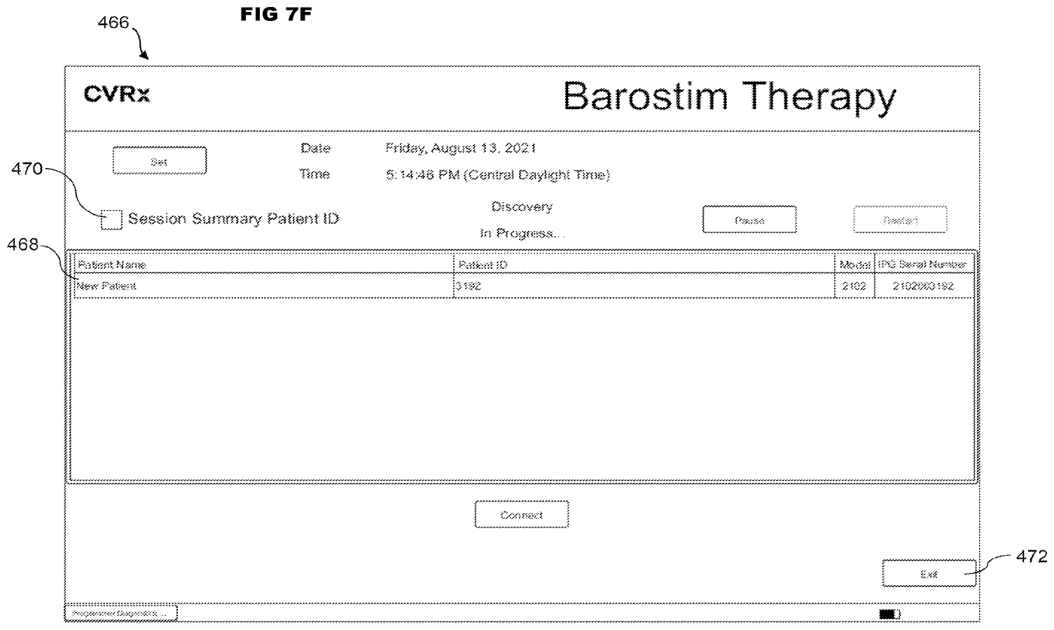

Turning now to the user interface of computing device 104, aspects of the user interface may be as depicted in FIGS. 7A-7F during various stages of the programming process, although other arrangements of the user interface are contemplated and within the spirit of the present disclosure.

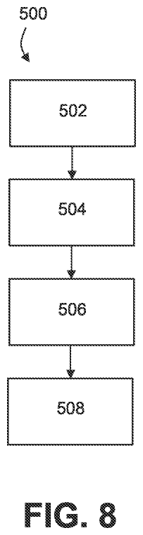

Referring also to FIG. 8, a method 500 of programming a baroreflex activation device is depicted according to embodiments. At step 502, the user is presented with the main operational page 400, as depicted in FIG. 7A. A header 402 of the main operational page 400 indicates patient status 404, which can include a patient ID 406 to maintain the anonymity of the patient, therapy status 408, battery status 410 of the implantable baroreflex activation system including a recommended replacement time and date, lead impedance 412 of any leads in use, and radio frequency (RF) signal strength 414. In embodiments, header 402 can persist across some or all pages of the user interface.

Main operational page 400 further includes therapy details 416, schedule 418, lead trends 420, and session notes 422. In embodiments having a touchscreen incorporated into the computing device, session notes 422 can expand to include a touch keyboard or keypad when selected.

Main operational page 400 provides a user with an overview of all necessary information while allowing the user to selectively explore additional details of the baroreflex activation therapy as needed. Main operational page 400 additionally provides reporting capabilities across sessions.

The user can stop all therapy from the main operation page 400 by selecting the master stop button 424. When therapy is stopped, therapy status 408 indicates that no therapy is being provided as shown in FIG. 7B, and the master stop button 424 is changed to a resume therapy button 426.

At step 504, the user can select Edit and Test button 428 to change parameters of baroreflex therapy. When Edit and Test button 428 is selected, the user can be presented with parameter adjustment page 430 as shown in FIG. 7C. On parameter adjustment page 430 the user is presented with parameter adjustment options for one or more leads including pulse width, amplitude, and frequency. The user can then test the compliance of any adjusted parameters by selecting the Test Now button 436. Pressing Test Now button 436 sets the voltage rails of the baroreflex activation device to the max, measures voltage at the end of the constant current pulse, and adds a margin to it. If the margin is above what the compliance or device would allow the parameters are deemed non-compliant because the device could not deliver the required voltage for therapy consistently and reliably such that therapy would be safe. Thus, compliance refers to the device's ability to set its voltage rails to deliver constant current. In embodiments, a compliance measurement is stored in a memory of the baroreflex activation device four times each day for troubleshooting and diagnosing capabilities.

Additionally, on parameter adjustment page 430 the user may select gear parameter adjustment button 440 to be presented with the parameter adjustment view shown in FIG. 7C. The user can select home button 438 to be presented with a slider 434 for can easily adjusting amplitude 432, as shown in FIG. 7D. Parameter adjustment page 430 further includes patient response log 442 that can include records, such as example record 444. Selecting pencil button 446 on a record allows a user to change to input notes (separate from session notes 422). To navigate back to main operational page 400 from parameter adjustment page 430 the user can select Exit without Saving button 448 or Save Selected Log Entry as Therapy 1 button 450.

At step 506, the user can select lead trends details button 474 to navigate to lead impedance trend page 452, as depicted in FIG. 7E. Lead impedance trend page 452 includes detailed trend graph 454, historical trend graph 456, and navigational buttons 458. Both detailed trend graph 454 and historical trend graph 456 can display the lead impedance trend for one or two implanted leads. In embodiments, lead impedance is tested once a day for each implanted lead and is stored in a memory of the baroreflex activation device. In embodiments, a user can select a portion of historical trend graph 456 to change the display of detailed trend graph 454 such that the highlighted portion 460 of historical trend graph 456 is represented by detailed trend graph 454. To navigate back to main operational page 400 from lead impedance trend page 452 the user can select Previous button 462.

At step 508, the user can select End Session button ending the communication with the current device and to navigate to patient selection page 466 as depicted in FIG. 7F. Patient selection page 466 enables a user to view a list of patients 468 with which the programmer can connect. In embodiments, Session Summary Patient ID box 470 can enable anonymous session reports by including only IPG SN on the session report. To exit the application from the patient selection page 466 the user can select Exit button 472.

It should be understood that step 504, step 506, and step 508 of method 500 are optional and can be completed in any order so long as the present teachings remain operable.

In embodiments, the programmer software uses obfuscation and encryption of all software constants to prevent tampering, reverse engineering, and discovery of proprietary programming algorithms.

In some embodiments, the present disclosure may comprise a kit which includes an implantable baroreflex activation system, a programmer, and a set of instructions recorded on a tangible medium for implanting, programming and/or operating the system. The contents of the kit may be provided in one or more hermetically sealed and sterilized packages. The implantable baroreflex activation system includes an implantable pulse generator within a hermetically sealed housing, a baroreflex activation device in the form of an electrode structure coupled to the pulse generator via a lead, and configured to be implantable proximate a baroreceptor of a patient. The programmer is as described elsewhere herein, and is configured to communicate with the implantable pulse generator for the purpose of programming and/or adjusting one or more operating parameters of the baroreflex activation system for the purpose of delivering a baroreflex activation therapy to a patient. The instructions may be recorded on a tangible medium, or indications may be provided linking a user to electronically accessible instructions.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112 (f) are not to be invoked unless the specific terms “means for” or “step for” are recited in a claim.

1. A programming system for programming a therapy having at least two programmable operating parameters for an implantable baroreflex stimulation system that is configured to deliver the therapy to a patient, the programming system comprising:
a programing device communicatively coupled to a computing device and the implanted baroreflex stimulation system, the programming device having a body pivotally coupled to a base, the base including:
a protrusion, and
a button configured to, when pressed, apply force adjacent to and towards the protrusion; and
a case configured to receive the computing device, the case having one or more receiving components configured to removably receive the protrusion, such that the case and the programming device are coupled until the button is pressed.

2. The programming system of claim 1, wherein the programming device further includes an aperture configured to allow the programming device to be hung.

3. A method of programming a therapy having at least two programmable operating parameters for an implanted baroreflex stimulation system, the implanted baroreflex stimulation system configured to deliver the therapy to a patient, the method comprising:
presenting, via a user interface of a computing device, a main page displaying patient information, therapy status, battery status, lead impedance, radio frequency (RF) signal strength, a graph of lead impedance trend of the implanted baroreflex stimulation system, and the at least two programmable operating parameters of the therapy;
displaying, via the user interface, a second page including an option to adjust each of the at least two programmable operating parameters;
receiving user input to adjust one or more of the at least two programmable operating parameters;
determining compliance of the implanted baroreflex stimulation system operating with the adjusted one or more programmable operating parameters; and
in response to a determination of compliance, programming the implanted baroreflex stimulation system to use the adjusted one or more programmable operating parameters.
